(19) United States
(12) Patent Application Publication
(10) Pub. No.: US 2004/0087973 A1
(43) Pub. Date: May 6, 2004

(54) Multiple-hair removal device and method of use

(76) Inventor: Golnaz Shobeiri, Columbia, MD (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/645,803

(22) Filed: Aug. 22, 2003

Related U.S. Application Data
(60) Provisional application No. 60/405,844, filed on Aug. 26, 2002.

(51) Int. Cl. A61B 17/50

(52) U.S. Cl. 606/133

(57) ABSTRACT

A hair removal device including one or more spring-like mechanisms attached at either end to grip handles connected at a pivot point or that includes handles at the ends. In one variation, the user squeezes the handles open, expanding the spring-like mechanisms, such that hair slips into the mechanism's openings. The user then returns the handles to their resting state, causing the mechanisms to grasp the hair. The device is then pulled away from the skin, plucking the entrapped hairs. Another variation is a floppy cord of spring-like mechanisms with grip handles at both ends that a user pulls in opposing directions, allowing hairs to enter the resulting openings. Entrapped hairs are plucked when the tension is released from the device, and the device is pulled from the skin. The second variation thus allows for more precise hair removal in smaller or difficult to access areas than the first variation.

This application claims priority from U.S. Provisional Application Serial No. 60/405,844 filed Aug. 26, 2002. The entirety of that provisional application in incorporated herein by reference.

### BACKGROUND OF THE INVENTION

1. Field of the Invention

The present invention relates to a device to remove hairs.

2. Background of the Technology

Well known hair removal techniques have many disadvantages. Shaving requires frequent repetition in order to maintain smooth skin and to prevent visible hair growth. Drawbacks of shaving including safety concerns for cutting of the user; in addition, shaving can only be used with certain parts of the body (e.g., face) with assistance such as with use of a mirror. Waxing often requires professional assistance that can be expensive and inconvenient or can be difficult to self apply for certain body areas (e.g., the back). Waxing also has other drawbacks. For example, wax sticks to the skin, and the skin must be cleaned after waxing.

With conventional tweezers, a user can only remove one hair at a time, and such detailed plucking often requires a good eye and a mirror. Tweezers, with their sharp ends, can also be dangerous. Additionally, with conventional tweezers (as well as with waxing techniques), one often has to wait for the regrowth of unwanted hair before attempting to remove the hair again.

### DESCRIPTION OF THE RELATED ART

U.S. Pat. No. 4,923,460 to Amit discloses a hair removal device. The user removes hair by rolling a roughened coil wire across the skin, causing some hairs to get caught in and plucked by the loops of the coil. Because the coil itself is not expanded or compressed by the user, one drawback for the effectiveness of the device is that its effectiveness depends upon whether unwanted hair gets caught in the coils, as the device is merely rolled across the skin.

U.S. Pat. No. 6,159,222 to Yiu discloses an electrically or manually operated hair removal device. The device consists of an expanded coiled spring held between two arms. The user places the device over the unwanted hair and applies force to compress the spring, which grasps the hair for removal. One problem is that, because the spring is stretched open in the device's resting state, the user must apply force to close the device while simultaneously pulling the hair.

U.S. Pat. No. 5,163,288 to Doley discloses a rotary head multi-spring hair removal device. Springs mounted on the rotary head open and close to grip and pluck hair as they rotate. This device is designed to remove a large area of hair. Consequently, one problem with this device is that the user does not have precise manual control over which hairs are removed. Another problem is that the device requires an adequate power source.

### SUMMARY OF THE INVENTION

The present invention provides multiple-hair removal devices that offer the user more control and efficiency than the prior art. The present invention is structurally simpler than much of the prior art and is consequently easier and less expensive to manufacturer, easier to operate, and easier to maintain. Further, the present invention does not leave a residue or require cleaning of the skin after use, is safe to use, and allows use without the benefit of viewing the area for which hair is being removed (e.g., it can be used with the eyes closed and on areas that cannot be viewed by the user), as well as allowing use when it is inconvenient to view the area (e.g., the present invention may be used by a person while driving).

An embodiment of the present invention comprises a multiple-hair removal device having grip handle portions comprising two arms joined by a bolt or rivet. Held between the two arms are one or more rows of spring-like mechanisms, composed, for example, of metal or other material, that regain their initial shape after being expanded and then released. Each spring-like mechanism is attached to the handles by a screw or other attaching feature located at a lever point. In operation, when the user compresses the lower portions of the handles together, the upper portions of the handles pull apart and expand the spring mechanisms via the lever point. The user can place the expanded spring mechanisms over hairs to be removed, causing the hairs to become entrapped in the openings of the spring mechanisms when the user releases the handles to close the spring mechanism. The user can then pull the device away from the skin surface, thereby plucking the hairs.

Another embodiment of the present invention comprises a multiple-hair removal device that includes a cord of one or more spring-like mechanisms attached to handles at either end of the cord. In operation, the user pulls the handles in opposing directions to expand the spring-like mechanisms and create openings in which unwanted hairs may be trapped. The user then allows the cord to relax to its resting position, such that hairs to be removed are clenched by the spring-like mechanisms and may be plucked by pulling the device away from the skin. The device can be used for removing hair from the upper lip, the chin, the legs, and other such places.

Additional advantages and novel features of the invention will be partially set forth in the description that follows, and will also become apparent to those skilled in the art upon examination of the following or upon learning by practice of the invention.

### BRIEF DESCRIPTION OF THE DRAWINGS

FIG. 1 is a perspective view of the grip-handled multiple-hair removal device, in accordance with one embodiment of the present invention.

FIG. 2 is a front view of the cord multiple-hair removal device, in accordance with one embodiment of the present invention.

### DETAILED DESCRIPTION

The present invention provides hair removal devices and techniques that are more convenient and efficient in use than the prior art. An embodiment of the present invention comprises a hair removal device with grip handles housing spring-like mechanisms that the user can expand and compress to entrap and pull away hairs. Another embodiment includes a cord spring-like mechanism that the user can expand and compress to entrap and pull away hairs in a more precise manner, or for removing hair in places harder to reach with the first embodiment.

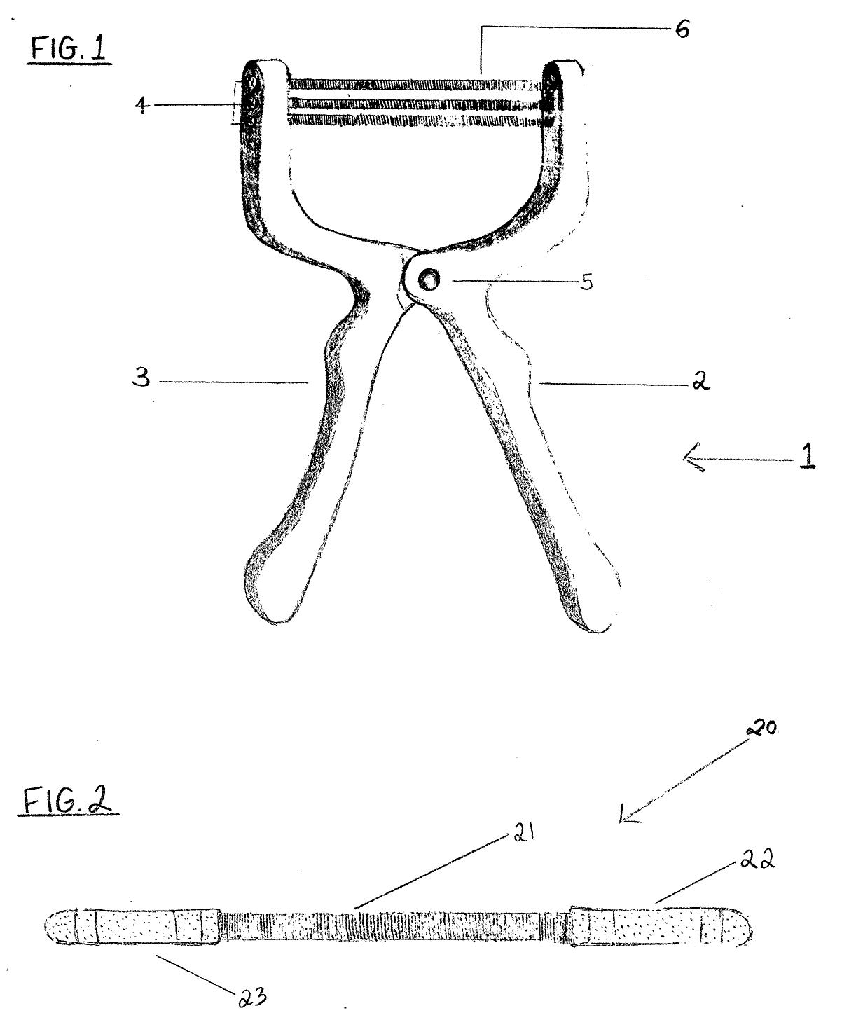

A first embodiment of the invention, as shown in FIG. 1, comprises a handle portion 1 that includes two arms 2, and 3, joined together at a pivot point 5, such as by a bolt or rivet-like feature, and multiple rows of spring-like mechanisms 6 connecting the upper portion of each arm handle 2, 3, as shown in FIG. 1. A second embodiment of the invention is shown in FIG. 2 and comprises a bendable, spring-like mechanism 21, with handles 22, 23 at both ends of the spring-like mechanism 21. This embodiment generally can be used for more precisely removing hairs. The following description contains detailed references regarding each of the embodiments shown in FIGS. 1 and 2.

As shown in FIG. 1, in one embodiment, handles 2, 3 comprise two solid or semi solid (e.g. perforated or hollow) arm pieces that are designed to be held and gripped in the hand. In the embodiment shown, handles 2, 3 are pivotally connected, such as by a bolt or rivet 5. In one embodiment, handles 2, 3 comprise a lightweight material, such as plastic, that is ergonomically designed to fit comfortably in the hand. Each handle is of a suitable length, and in one embodiment is approximately 6½ inches long and approximately ½ an inch wide.

The handles 2, 3 are connected by one or more rows of spring-like mechanisms 6 that, when pulled simultaneously in opposing directions, biasedly oppose such pulling, and that regain their initial shape when released. The material composing the mechanism 6 includes metal or another similar elastic material and design such that, when extended, form openings large enough to entrap hairs, and when compressed, the mechanism 6 interlocks sufficiently to grasp and catch hairs into the openings of the material. The material has a spring-like quality, though is not necessarily coil-shaped. Each row of the spring-like mechanism 6 has two ends that are each connected to one of the top portions of handles 2, 3, via, for example, fastening mechanisms 4, such as screws or similar type connectors. Fastening mechanisms 4 securely attach spring-like mechanism 6 to handles 2, 3, such that when the bottom portion of the handles 2, 3 are squeezed together towards each other in the hand, the result is that, via levering about pivotal point 5, the top portion of handles 2, 3 are separated from one another, and the spring-like mechanism 6 expands. When the bottom portions of handles 2, 3 are allowed to spread apart again (via biased spring-like mechanism 6), the top portions move closer together to the original position, as depicted in FIG. 1, causing the spring-like mechanism 6 to regain its initial, unexpanded length. In one embodiment, pivot point 5 allows the two handles 2, 3 to move in a scissor-like fashion when pulled together and released apart by the hand.

In order to remove hair with the grip-handled multiple-hair removal device depicted in FIG. 1, the handles 2, 3 are compressed, such that the upper portion of the handles expand and pull open the spring-like mechanism 6. Then, while squeezing the handles together, the device is placed against the skin surface with the unwanted hair, such that hair is able to fit into the tiny openings created by expansion of the spring-like mechanism 6. The spring-like mechanism 6 thereby allows the entrapment of one or more hairs. The user then releases handles 2, 3, such that the lower portions of the handles 2, 3, as shown in FIG. 1, spread apart and the upper move together, allowing the spring-like mechanism 6 to compress and interlock any hairs within the openings formed by expansion of the spring-like mechanism 6. The final step in removing the unwanted hair from the skin surface involves pulling or rolling of the grip-handled device while the hair remains interlocked in the spring-like mechanism 6, such that the hair is plucked from the skin. The device can be used against both flat and curved skin surfaces, including, for example, the upper lip, the chin, and the legs.

FIG. 2 presents a front view of the cord multiple-hair removal device of a second embodiment of the present invention.

As shown in FIG. 2, the device 20 includes a spring-like mechanism 21, which, when pulled at both ends simultaneously, expands, and when such pulling is released, regains its initial, unexpanded length. The material is composed, for example, of metal or another similar elastic material having a shape that has openings large enough to receive hairs when expanded. The material has a spring-like quality, though it is not necessarily coil-shaped. Additionally, an embodiment of this invention could include multiple rows of spring-like mechanisms 21 to entrap hairs. In one embodiment, each end of the spring-like mechanism 21 is fitted with handles 22, 23, which are composed partially or wholly of plastic or another similar material that is easily gripped. In one embodiment, the handles 22, 23 are lightweight and somewhat roughened by slightly raised studs so as to provide the user with a better grip.

In order to more precisely remove unwanted hair, or for use in locations, for example, that are hard to reach using the first embodiment, one may use the cord multiple-hair removal device 20 depicted in FIG. 2. To remove hair from a skin surface, one uses both hands to grip each of handles 22, 23, then places the device against the skin surface, and pulls the handles apart so as to expand the spring-like mechanism 21. Once the spring-like mechanism 21 is expanded, one or more hairs are able to fit into the openings created by the expansion. The user then slowly releases the spring-like mechanism 21 to its resting position, such that the hairs in the openings are entrapped within the coils. Once the hairs are securely entrapped in the spring-like mechanism 21, the user may lift, pull, or roll the device away from the skin, thereby plucking the hairs from the skin.

Although specific embodiments of the invention have been described, these embodiments are not meant to limit the scope of the appended claims because further modifications will now suggest themselves to those skilled in the art.

All documents that are filed concurrently and are open to public inspection along with this specification are incorporated herein by reference. All the features disclosed in this specification, including accompanying claims, abstract, and illustrations, may be replaced by alternative features serving the same, equivalent, or similar purpose, unless expressly stated otherwise.

1. A hair removal device, comprising:
a first handle having a first end and a second end;
a second handle having a first end and a second end, the second handle being connected to the first handle via a pivoting mechanism; and
a spring-like mechanism coupled to the first handle at the first handle first end and coupled to the second handle at the second handle first end;
wherein the device is placeable in a first configuration in which the spring-like mechanism is unexpanded;
wherein the device is placeable in a second configuration in which the spring-like mechanism is expanded via drawing the first handle second end and the second handle second end together, the first handle first end and the second handle first end thereby being drawn apart via the pivoting mechanism, the first handle first end and the second handle first end thereby expanding the spring-like mechanism, such that at least one opening is formed in the spring-like mechanism;
wherein a hair is received in the formed at least one opening when the device is in the second position; and
wherein the hair is entrapped in the hair removal device upon the device being placed in the first position following receiving of the hair in the formed at least one opening.

2. The hair removal device of claim 1, wherein the entrapped hair is removed via pulling of the device.

3. The hair removal device of claim 1, wherein the entrapped hair is removed via sliding of the device.

4. The hair removal device of claim 1, wherein the entrapped hair is removed via rolling of the device.

5. The hair removal device of claim 1, wherein the first handle comprises a solid material.

6. The hair removal device of claim 5, wherein the solid material is plastic.

7. The hair removal device of claim 1, wherein the spring-like mechanism comprises a coiled spring.

8. The hair removal device of claim 1, wherein the spring-like mechanism is coupled to the first handle and the second handle via a connector.

9. The hair removal device of claim 8, wherein the connector is a screw.

10. A hair removal device, comprising:
a spring-like mechanism having first and second ends, the spring-like mechanism being configurable into an expanded configuration and an unexpanded configuration, wherein the mechanism is biased in the unexpanded configuration;
a first handle attached to the first end of the spring-like mechanism; and
a second handle attached to the second end of the spring-like mechanism.

11. The hair removal device of claim 10, wherein the spring-like mechanism comprises a spiral spring.

12. The hair removal device of claim 10, wherein at least one of the first and second handle comprises plastic.

13. The hair removal device of claim 10, wherein at least one of the first and second handle comprises a roughened surface.

14. The hair removal device of claim 10, wherein at least one of the first and second handle comprises a surface having raised studs.

15. The hair removal device of claim 10, wherein the device is usable to remove hairs by expanding the device to the expanded configuration via the first and second handles, receiving a hair in the spring-like mechanism while the mechanism is in the expanded configuration, and entrapping the hair within the spring-like mechanism by allowing the mechanism to return to the unexpanded configuration.

16. A method for hair removal using a hair removal device, the hair removal device including a first handle having a first end and a second end; a second handle having a first end and a second end, the second handle being connected to the first handle via a pivoting mechanism; and a spring-like mechanism coupled to the first handle at the first handle first end and coupled to the second handle at the second handle first end, the method comprising:
placing the device in a first configuration in which the spring-like mechanism is unexpanded;
placing the device in a second configuration in which the spring-like mechanism is expanded via drawing the first handle second end and the second handle second end together, the first handle first end and the second handle first end thereby being drawn apart via the pivoting mechanism, the first handle first end and the second handle first end thereby expanding the spring-like mechanism, such that at least one opening is formed in the spring-like mechanism;
receiving a hair in the formed at least one opening when the device is in the second position; and
entrapping the hair in the hair removal device upon the device being placed in the first position following receiving of the hair in the formed at least one opening.

17. The method of claim 16, wherein the hair is attached to skin, the method further comprising:
moving the device such that the hair is detached from the skin.

18. The method of claim 17, wherein moving the device includes:
drawing the device away from the skin.

19. The method of claim 17, wherein moving the device includes:
pivoting the device relative to the skin.

20. A method for removing hair using a hair removal device, the hair removal device including a spring-like mechanism having first and second ends, the spring-like mechanism being configurable into an expanded configuration and an unexpanded configuration, wherein the mechanism is biased in the unexpanded configuration; a first handle attached to the first end of the spring-like mechanism; and a second handle attached to the second end of the spring-like mechanism, the method comprising:
expanding the device to the expanded configuration via the first and second handles;
receiving a hair in the spring-like mechanism while the mechanism is in the expanded configuration; and
entrapping the hair within the spring-like mechanism by allowing the mechanism to return to the unexpanded configuration.
